(19) United States
(12) Patent Application Publication
(10) Pub. No.: US 2006/0127461 A1
(43) Pub. Date: Jun. 15, 2006

(54) Wound dressings containing an enzyme therapeutic agent

(76) Inventor: Stephen Bloor, Chorley (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/536,540

(22) PCT Filed: Dec. 5, 2003
(86) PCT No.: PCT/GB03/05296

§ 371 Date: Dec. 14, 2005

(30) Foreign Application Priority Data
Dec. 6, 2002 (GB) 0228554.2

(51) Int. Cl. A61K 38/44; A61L 15/00

(52) U.S. Cl. 424/445; 424/94.4

(57) ABSTRACT

The invention provides a wound dressing or implant comprising an enzymatic compound or reagent that is effective to reduce the concentration of lactate in an aqueous solution in contact with the dressing. Preferably, the compound is a lactate oxidase enzyme that converts lactate present in wound fluid as a consequence of wound hypoxia to pyruvate and hydrogen peroxide.

The present invention relates to the field of wound healing. More particularly, the present invention provides dressings and implants for use in the treatment of wounds that accelerate the healing process by decreasing the concentration of lactate in the environment of the wound.

Oxygen is a prerequisite for the formation of chemical energy within living cells. When a wound tissue becomes hypoxic, the tissue will preferentially use the glycolytic pathway to generate energy in the form of adenosine triphosphate (ATP), since the amount of oxygen is limiting.

Pyruvate is converted to lactate by lactate dehydrogenase, in the process generating two molecules of ATP per molecule of pyruvate hydrolysed. However, only a small fraction of the potential energy content of glucose is released by anaerobic conversion into lactate; much more energy can be released by the oxidative decarboxylation of pyruvate via the citric acid cycle.

Lactate is in effect a metabolic “dead-end” in the mammalian body, as it must be converted back into pyruvate before it can be metabolised. In mammals, this reaction is only performed in the liver. Consequently, in wounds, lactate concentrations often rise to levels which are detrimental to the healing process. In particular, the presence of large amount of lactic acid in the wound causes a severe drop in the pH of the wound and thus slows down the healing process; (the ideal pH for the healing process to take place in the wound is thought to be around 6.0). In addition, high levels of lactic acid upset the redox balance of the wound, and impair metabolic balance in other ways.

Currently preferred treatments to accelerate the healing of wounds involve a variety of wound dressings. Such dressings include absorbent wound dressings such as polyurethane foam dressings, bioabsorbable freeze-dried collagen sponges, the collagen-ORC (oxidized regenerated cellulose) freeze dried sponges known as PROMOGRAN (Registered Trade Mark), the collagen-alginate composite known as FIBRACOL (Registered Trade Mark), bioabsorbable polysaccharide or polypeptide biopolymers and simple medicated wound dressings. The latter types include INADINE (Registered trade mark), a slow release povidone iodine wound dressing, FLAMAZINE (Registered trade mark), a 1% silver sulfadiazine product and VARIDASE (Registered trade mark), which is a debriding agent containing streptokinase and streptodomase. Therapeutic pharmaceutical compositions are also used, such as IAMIN (Registered trade mark), a copper-peptide product, PROCUREN (Registered trade mark) and a natural platelet-derived wound healing composition.

Some research has been reported regarding the use of oxygen-containing compounds for use in the treatment of wounds. For example, Weiss & Evers (1988) Aktuelle traumatol. vol 18(5), pp 219-225 describe the use of tetrachlorodecaoxide (TCDO) in the treatment of complicated wounds. Hinz et al. (1984) Fortschr. med. vol 102(18), pp 523-528 describes the stimulation of wound healing by TCDO in a randomised double blind study.

There has also been a hypothesis proposed which suggests that raising oxygen tensions in a wound may aid a wound healing prognosis (Kuhne et al. (1985) Infection vol 13(2), pp 52-56). This paper describes a correlation between tissue oxygen tension, incidence of wound infection and disturbance of wound healing.

U.S. Pat. No. 4,507,285 describes stabilised activated oxygen in a matrix of chlorite ions and pharmaceutical compositions that contain stabilised activated oxygen. Such compositions are proposed to be useful for the purpose of stimulating oxygen metabolism in an organism, and the treatment of skin diseases and wound healing disorders.

U.S. Pat. No. 4,851,222 describes the use of an aqueous solution of stabilised oxygen within a matrix of chlorite ions to promote the regeneration of bone marrow.

International Patent Application WO91/08793 discloses a treatment system for wounds and other disorders wherein a flexible chamber is secured about the periphery of a wound and allows the introduction of a treatment fluid consisting of saline solution, antibiotics and anaesthetics. The maintenance of a wound in this solution accelerates the healing process.

However, all treatments reported to date are far from ideal. Very few treatments or dressings efficiently protect the wound from bacterial infection. Consequently, particularly in the case of chronic wounds, infection can slow or reverse the healing process. Furthermore, in most instances, the assessment of the metabolic state of a wound or the evaluation of the progress of wound healing requires costly and advanced techniques that require the attention of a skilled operative. Thus, in most cases, wounds heal largely through the unstimulated action of the body's immune system.

There thus remains a need for improved materials and methods for the treatment of wounds that accelerate their healing. There is also a need for a material that is capable of assessing the environment of a wound and that can respond in order to redress the metabolic balance of the wound towards that at which healing is enhanced.

According to the present invention there is provided a wound dressing or implant comprising an enzymatic compound or reagent that is effective to reduce the concentration of lactate in an aqueous solution in contact with the wound dressing or implant.

Wounds suitable for treatment using the dressings or implant of the present invention will be known to those of skill in the art and include burn wounds, incisional wounds, excisional wounds, tumours, skin diseases and other skin or superficial disorders, and in particular chronic wounds such as venous ulcers, pressure sores, decubitus ulcers, herpes eruptions and chemical ulcers.

The choice of whether to use a wound dressing or implant to treat a wound will be matter of choice for the person of skill in the art and will depend on the nature of the wound. Implants will be of particular use in accordance with the present invention in deep or puncture wounds whereas a flat dressing is not able adequately to cover the total surface area of the wound.

The wound dressing may comprise a solid material into or onto which an enzymatic compound or reagent may suitably be incorporated. For example, suitable dressings include absorbent wound dressings such as nonwoven fabrics and foams (e.g. polyurethane foams, for example as described in EP-A-0541391. In other embodiments, the enzymes are dispersed in or on solid bioabsorbable materials such as collagen sponges, polylactide/polyglycolide structures, collagen-alginate composite dressings for example as described in U.S. Pat. No. 4,614,794, collagen-ORC composite structures as described in EP-A-0918548, or other bioabsorbable polysaccharide or polypeptide biopolymers. In yet other embodiments, the enzyme is dispersed in a suitable gel or ointment for topical administration to a wound. Other wound dressings will be known to those of skill in the art and will comprise any solid dressing to the surface of which a suitable compound or reagent may be adsorbed or chemically bound, or into which a suitable compound or reagent can be incorporated for sustained release.

Any enzymatic compound or reagent may be associated with the wound dressing or implant of the present invention that is capable of causing a decrease in the concentration of lactate in an aqueous solution under physiological conditions of temperature, pH, lactate concentration, oxygen, CO₂ concentration and so forth, such as is found in the environment of a wound. Preferably, the compound or reagent comprises an enzyme.

The activity of the dressings can be specified in terms of activity units per gram of the dressing. One unit will remove 1.0 μmol of L-lactate per minute at pH6.5 at 37° C. Thus, for example, one unit of lactate oxidase activity is the amount needed to oxidize 1.0 μmol of L-lactate to pyruvate and H₂O₂ per minute at pH6.5 at 37° C. Preferably, the activity (e.g. lactate oxidase activity) of the dressings is from about 0.001 units/g to about 100 units/g, more preferably from about 0.01 units/g to about 10 units/g, and most preferably from about 0.1 units/g to about 1 unit/g.

Most preferably, the compound or reagent comprises a lactate oxidase enzyme. Lactate oxidase may be derived from any organism or may be partially or wholly synthetic. Suitable lactate oxidase species are present in both prokaryotes and eukaryotes. From the point of view of expense, prokaryote-derived enzymes will be preferred, although eukaryote enzymes, preferably mammalian or human, are less likely to cause immunogenic reactions in the wound site. Human lactate oxidase is most preferable.

The activity of pure freeze-dried lactate oxidase is about 20 to 40 units/mg. Preferably, each gram of the dressings according to the present invention contains from about 0.1 ng to about 1 mg of lactate oxidase, more preferably from about 1 ng to about 100 ng of lactate oxidase.

Lactate oxidase enzyme that have been engineered to possess advantageous properties over the wild type species may also be used according to the present invention. In particular, enzymes may be modified by site-directed mutagenesis to accelerate the rate at which they metabolise lactate or to reduce the immunogenicity of the protein.

Lactate oxidase acts to catalytically convert lactic acid into pyruvic acid that will diffuse into the environment of the wound, where it may be utilised as an energy source by the cells of the wound through its oxidative carboxylation as part of the citric acid cycle. The availability of this extra energy source will allow the cells of the wound to grow more quickly. Furthermore, the pH of the wound environment will increase as the lactic acid concentration in the wound falls.

The oxygen needed for lactate oxidase reaction comes from the environment of the wound and from the atmosphere itself. The hydrogen peroxide generated as a by-product of this reaction of lactate oxidase with oxygen may spontaneously decompose to release oxygen back into the wound.

The hydrogen peroxide may also be beneficial to the wound healing process. For example, hydrogen peroxide is a bactericidal agent, acting to inhibit the growth of microbes on the wound surface, thereby minimising the risk of development of clinical infections in the wound. As a by-product of this effect, this chemical acts to minimise the build-up of chemical odours developing from microbial growth in the wound.

In use, the higher the lactate concentration in the wound, the greater the activity of lactate oxidase in the wound dressing or implant that will result. Consequently, the system is self-regulating.

Additional compounds may also be coupled to the device of the present invention. For example, a compound can be used that accelerates the reduction of H₂O₂ into H₂O and molecular oxygen. For example, a suitable enzyme that catalyses this process is the catalase enzyme. This reaction is set out below.

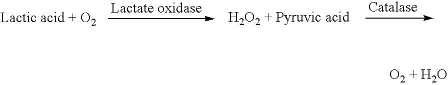

The use of catalase as a coupled enzyme has the advantage that local oxygen levels in the wound environment may be boosted, causing a concomitant increase in growth of cells in the environment of the wound. Catalase enzyme may be obtained from any source, as discussed above for lactate oxidase. Potato homogenate is a particularly good source of catalase. Catalase activity is generally defined such that one unit will decompose 1.0 μmol of H₂O₂ per minute at pH 7.0 at 25° C., while the H₂O₂ concentration falls from 10.3 to 9.2 mM. Preferably, the catalase activity per gram of the wound dressings of the present invention is within one of the preferred ranges specified above for the lactate oxidase activity. The activity of commercially available catalase varies from about 1000 units/mg to about 50,000 units/mg. It follows that the amount of catalase used to make the wound dressings of the invention is preferably about 0.01 ng to about 10 ng/gram of the dressing.

Indicator systems that are responsive to the concentration of hydrogen peroxide in a wound may also be associated with the wound dressing or implant of the present invention, whereby the indicated concentration of H₂O₂ produced by the reaction between lactate oxidase and lactic acid gives an indication of the concentration of lactate initially present in the wound environment. This will give a physician useful information about the metabolic condition of the wound, for example an indication of the degree of hypoxia.

The indicator systems comprise a redox indicator compound, which is usually activated by a peroxidase enzyme in the presence of hydrogen peroxide.

Preferably, the indicator compound is a chromogenic compound. Suitable chromogenic substrates suitable as coupled indicators of lactate concentration include the following, along with the colour produced upon oxidation by H₂O₂. ABTS (2,2′-azino-bis-(3-ethylbenzthiazoline-6-sulphonic acid) [green]; OPD (o-phenylenediamine) [orange]; TMB (3,3′-5,5′-tetramethylbenzidine) [blue]; O-dianisidine [orange]; 5AS (5-aminosalicylic acid) [brown]; DAB (3,3′-diaminobenzidine) [brown]; AEC (3-amino-9-ethylcarbazole) [blue]; 4C1N (4-chloro-1-naphthol) [blue]. All of these indicator compounds are available from Sigma Chemical Company.

For most of the above indicator compounds, a means of oxidation of the compound must also be present in the dressing or implant. Any means of oxidation may be used that can be coupled stoichiometrically to the amount of hydrogen peroxide present in the wound.

For example, a peroxidase enzyme may be incorporated into the device, so causing the oxidation of an indicator compound. This reaction is shown below:

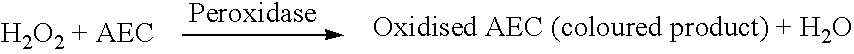

Preferably, the means of oxidation of the indicator compound comprises a peroxidase enzyme, more preferably horseradish peroxidase. Suitable concentrations of peroxidase enzyme and indicator can readily be determined by the person skilled in the art.

The enzyme agent is preferably bound to the material of a solid wound dressing or implant by any suitable means that ensures that the enzyme is not able to migrate from the material into the wound. The solid substrates of the wound dressings or implants of the invention may comprise amine, hydroxyl, sulfydryl, carbonyl or active hydrogen reactive chemistries. Consequently, preferred methods of attachment of the enzyme will comprise strong links such as covalent linkages, or use of binding pairs such as biotin and streptavidin. Preferably, the enzyme is bound to the material by a covalent linkage. Similarly, any other compounds whose presence is necessary for coupled reactions will be attached to the material in a similar way.

Covalent linkage of enzymes and indicators onto a solid wound dressing or implant material can preferably be achieved through the use of commercially available cross-linking reagents. The following reagents may be used to link one enzyme to a device or two enzymes to each other and then to a device: formaldehyde, cyanogen bromide, carbonyl diimidazole, carbodiimides, maleimide, epoxy (bisoxirane) activation, divinyl sulphone and hexamethyl diisocyanate (HMDI). Other suitable methods of cross-linking will be known to those of skill in the art. Suitable methods of incorporation of active agents into the material of the wound dressing or implant will be clear to those of skill in the art. In most cases, compounds or reagents will be included in the manufacture of the device so that they become entrapped in the device structure during the manufacturing process. For example, enzymes and (optionally) indicators may be included in a collagen or collagen-alginate or collagen-ORC slurry prior to freeze drying in a process similar to that used in U.S. Pat. No. 4,614,794 or EP-A-0918548, the entire contents of which are expressly incorporated herein by reference.

Similarly, enzymes and (optionally) indicators can be included in the manufacture of foam dressings, for example the polyurethane foam described in EP-A-0541391, by inclusion during the foam generation steps, so that the enzymes and indicators become entrapped within the foam structure. In certain preferred embodiments, the wound dressing or implant comprises a semi-permeable wound contacting top sheet such as dialysis membrane type material that retains added enzymes and indicators, but which allows the free transfer of wound fluid and metabolites from the wound into the dressing and vice versa.

The wound dressing or implant according to the present invention may also contain a medicament. Suitable medicaments will be well known to those of skill in the art and include antiseptics, such as povidone iodine or silver sulfadiazine; antibiotics such as enthromycin, neomycin, bacitracin, gentamycin, framycetin, thyrotrycin, polymyxin B, gramicidin, fusidic acid, chloramphemicol, tetracycline and its derivatives, minocycline chlortetracycline, hydrochloride, meclocyclin, penicillin and its derivatives, ampicillin or a cephalosporin; steroidal anti-inflammatories such as hydrocortisone, betamethasone, dexamethasone, prednisolone, and their derivatives; non-steroidal anti-inflammatories such as indomethacin, ketoprofen, ibuprofen and diclofenac; anaesthetics such as cocaine, benzocaine, procaine or lignocaine; analgesics such as aspirin; and anti-oxidants such as Vitamin E, Vitamin C, Zinc, selenium or cysteine.

The wound dressing or implant of the present invention may be in the form of a diagnostic sheet as disclosed in EP-A-0864864. For example, the wound dressing may be in the form of an absorbent sheet having impregnated therein or bound thereto a lactate oxidase, horseradish peroxidase, and a chromogenic redox indicator. If this sheet is contacted onto a large area wound, the intensity of colour developed on the sheet will map the concentration of H₂O₂ over the wound surface, and will thereby give a map of lactate concentration (i.e. hypoxia) over the surface of the wound. Individual regions of hypoxia within a larger wound can thereby be identified, and treated appropriately.

The present invention also provides the use of an enzymatic compound or reagent that is effective to reduce the concentration of lactate in aqueous solution for the preparation of a dressing or implant for the treatment of wounds. Preferably, the dressing or implant is as described above in relation to the first aspect of the invention. Preferably, the wound is a chronic wound such as a venous ulcer, a pressure sore or a diabetic ulcer.

According to a further aspect of the present invention there is provided a method of treating a wound in a mammal comprising applying to the wound a wound dressing or implant comprising an effective amount of an enzymatic compound or reagent that is effective in reducing the concentration of lactate in an aqueous solution.

Various aspects and embodiments of the present invention will be illustrated in the following prophetic examples. Further aspects and embodiments of the present invention will be apparent to those skilled in the art.

### EXAMPLE 1

### Hydrogen Peroxide Generating Dressing

To a collagen/calcium alginate slurry (90 parts collagen: 10 parts alginate, 1% w/v solids, prepared as described in U.S. Pat. No. 4,614,794, was added lactate oxidase in an amount of 0.01 unit/part by weight of collagen (Sigma Chemical Company; lactate oxidase from Pediococcus species) followed by HMDI (2% w/v). The mixture was agitated until mixing was achieved.

The slurry was poured into a container and freeze-dried overnight. The resulting collagen/alginate sponge pad contains immobilised lactate oxidase enzyme, which when exposed to wound fluid containing lactic acid generates hydrogen peroxide as a bacteriocide and wound cleanser.

### EXAMPLE 2

### Pyruvic Acid Generating Dressing

To a collagen/calcium alginate slurry (90 parts collagen: 10 parts alginate, 1% w/v solids prepared as described in U.S. Pat. No. 4,614,794 was added a lactate oxidase/catalase conjugate (prepared by incubation of lactate oxidase and catalase (Sigma Chemical Company), in an amount of 0.01 units each per part of collagen, with formaldehyde (1%, 1 hour), followed by removal of the excess formaldehyde by dialysis). Then added HMDI (2% w/v) and agitated until mixing was achieved.

The slurry was poured into a container, freeze dried overnight. The resulting collagen/alginate sponge pad contains immobilised lactate oxidase and catalase enzymes, which when exposed to wound fluid containing lactic acid generate pyruvic acid.

The pyruvic acid may be used by the wound as an alternative energy source. The hydrogen peroxide will be removed by the presence of catalase and will generate oxygen species in the wound that will also accelerate energy generation.

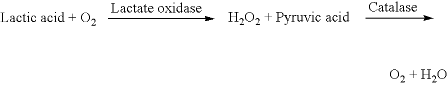

### EXAMPLE 3

### Dressing Indicating Wound Status

To a collagen/calcium alginate slurry (90 parts collagen: 10 parts alginate, 1% w/v solids) were added add lactate oxidase/peroxidase conjugate (prepared by incubation of lactate oxidase and peroxidase (Sigma Chemical Company; horse radish peroxidase), in an amount of 0.01 units each per part of collagen, with formaldehyde (1%, 1 hour followed by removal of excess formaldehyde by dialysis). Added HMDI (2% w/v) and 3-amino-9-ethylcarbazole (AEC) and agitated until mixing was achieved.

The slurry was poured into a container, and freeze-dried overnight. The resulting collagen/alginate sponge pad contains immobilised lactate oxidase and peroxidase enzymes, together with a redox indicator, which when exposed to wound fluid containing lactic acid will generate a colour change. The intensity of colour generated will be proportional to the concentration of lactic acid in the wound and will be indicative of the oxygen and metabolic status of the wound environment. For example, high lactic acid concentrations will indicate low oxygen tensions and a stressed environment.

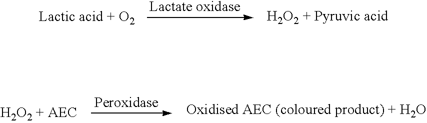

### EXAMPLE 4

An ointment containing lactate oxidase and suitable for topical administration to a wound such as a venous ulcer, decubitus ulcer or pressure sore is prepared by mixing the following ingredients in the following percentages by weight:

Freeze-dried lactate oxidase (Sigma) 0.005%

Hydroxyethyl Cellulose 0.35%

Carboxymethyl Cellulose 3.00%

Propylene Glycol 25.00 g

Sodium Chloride 0.30%

Distilled Water qs to 100%

The ointment is entirely wound-friendly and noncytotoxic, and can be applied to the chronic wound surface at regular intervals until wound healing is achieved.

The present invention has been described with reference to specific embodiments. However, this application is intended to cover those changes and substitutions which may be made by those skilled in the art without departing from the scope of the appended claims.

1. A wound dressing or implant comprising an enzymatic compound or reagent that is effective in reducing the concentration of lactate in an aqueous solution in contact with said wound dressing.

2. The wound dressing or implant according to claim 1, wherein the compound or reagent is bound to the surface of a solid wound dressing material.

3. The wound dressing or implant according to claim 1, wherein said compound or reagent comprises an enzyme.

4. The wound dressing or implant according to claim 3, wherein said enzyme comprises lactate oxidase.

5. The wound dressing or implant according to claim 1 additionally comprising a compound capable of reducing H₂O₂ to H₂O and molecular oxygen.

6. The wound dressing or implant according to claim 5 wherein said compound capable of reducing H₂O₂ to H₂O and molecular oxygen is a catalase enzyme.

7. The wound dressing or implant according to claim 1 additionally comprising a chromogenic redox indicator compound or system for indicating the presence of H₂O₂.

8. The wound dressing or implant according to claim 7 wherein said indicator compound or system comprises a compound selected from the group consisting of 2,2′-azino-bus-(3-ethylbenzthiazoline-6-sulphonic acid) (ABTS); o-phenylenediamine (OPD); 3,3′-5,5′-tetramethylbenzidine (TMB); 0-dianisidine; 5-aminosalicylic acid (5AS); 3,3′-diaminobenzidine (DAB); 3-amino-9-ethylcarbazole (AEC) and 4-chloro-1-naphthol (4CIN).

9. The wound dressing or implant according to claim 7, wherein said indicator system comprises a peroxidase enzyme.

10. The wound dressing or implant according to claim 1 comprising a solid material with amine, hydroxyl, sulfydryl, carbonyl or active hydrogen reactive chemistries.

11. The wound dressing or implant according to claim 1 comprising a proteinaceous, carbohydrate, plastic or polyurethane material.

12. The wound or implant according to claim 1 which is a solid dressing comprising a solid substrate having the enzymatic compounds or reagents dispersed thereon or therein.

13. The wound dressing or implant according to claim 1 which is a semisolid ointment for topical application comprising a hydrogel selected from cellulose derivatives, hydroxyethyl cellulose, hydroxymethyl cellulose carboxymethyl cellulose, hydroxypropylmethyl cellulose and hydrogels containing polyacrylic acid and mixtures thereof.

14. The wound dressing or implant according to claim 1 additionally comprising a medicament.

15. The wound dressing or implant according to claim 1 additionally comprising one or more immunological or other binding partners for one or more molecules present in a wound fluid.

16. Use of an enzymatic compound or reagent that is effective to reduce the concentration of lactate in an aqueous solution for the preparation of a dressing or implant for the treatment of wounds.
